(19) United States
(12) Patent Application Publication
(10) Pub. No.: US 2013/0255680 A1
(43) Pub. Date: Oct. 3, 2013

(54) Nasal Insert Impreganted with an Aromatherapy Oil

(71) Applicant: George VOLGYESI, Willowdale (CA)

(72) Inventor: George VOLGYESI, Willowdale (CA)

(21) Appl. No.: 13/430,833

(22) Filed: Mar. 27, 2012

(51) Int. Cl. A61M 15/08; A61M 16/14

(52) U.S. Cl. 128/204.13; 128/207.18

(57) ABSTRACT

This invention discloses a novel nasal insert which reduces the effect of upper airway resistance syndrome. The insert comprises a short, hollow, flexible tube with the central portion of one side being removed. The insert is inserted into each nostril to allow increased airflow and reduce the work of breathing. The efficiency of the insert may be increased by its impregnation with aroma-therapy essential oils.

### SUMMARY OF THE INVENTION

This invention relates to a novel nasal insert which reduces the effect of upper airway resistance syndrome.

### BACKGROUND OF THE INVENTION

Although one would think that we should take our ability to breathe while sleeping for granted, problems can occur anywhere from air entering our nose to the pipes that carry air into our lungs. During sleep, a human breathes through the nose. However, there are several reasons why some people find it difficult to breathe through the nose during sleep. The shape of the nostrils, nasal congestion, allergies or a deviated septum may increase nasal resistance thus leading to UARS. The septum separates the two nasal cavities and is ideally situated at the center of the nose equally separating the two sides. A deviated septum occurs when the septum inside the nose is displaced to one side increasing the resistance to airflow.

Upper airway resistance syndrome or UARS is a recently described form of sleep disordered breathing (hereinafter referred to as SDB) in which repetitive increases in resistance to airflow within the upper airway lead to brief arousals and daytime sleepiness and fatigue. In addition to these problems, patients have also complained of difficulty in concentration, morning headaches, impotence, difficulty in sleeping and restless sleep. Recently, UARS has been also linked to many somatic, psychiatric, or psychosomatic conditions, including attention deficit disorder or attention deficit hyperactivity disorder, fibromyalgia, and chronic insomnia.

There is no actual cessation of breathing, but patients with UARS suffer with increased airway resistance, which increases the work of breathing which then leads to arousal episodes and ultimately to excessive daytime sleepiness (hereinafter referred to as EDS). The most likely stimulus for arousal is the level of negative intrathoracic pressure mediated by mechanoreceptors in the upper airway. UARS may also occur in the absence of clinically significant snoring and may be an occult cause of EDS.

UARS events are noted to be to be typically short (one to three breaths in duration) and have been termed respiratory effort-related arousals with no evidence of oxygen desaturation. UARS affects men and women about equally and is so new, not all doctors are familiar with it, and many sleep clinics don't use techniques capable of identifying subtle changes in breathing patterns. The prevalence of UARS in the general population has been estimated to be as high as 10% to 15%.

If the breathing problem occurs in the upper airways (e.g. the nose) it is called ‘nasal resistance.’ Nasal resistance can contribute to sleep disordered breathing by causing upper airway resistance syndrome (UARS), snoring and obstructive sleep apnea (OSA). Upper airway resistance syndrome and obstructive sleep apnea are part of the spectrum of sleep disordered breathing. When it is mild, it manifests as UARS, and most standard sleep studies will not detect it unless they are specifically looking for it. When the blockage of air is more severe, as often occurs lower down in the airway, it manifests as sleep apnea. Paradoxically, the symptoms of UARS may be more severe than those of sleep apnea and are more likely to mimic CFS and fibromyalgia. Just as it is common to find that people with CFS/FMS have sleep disordered breathing, the reverse is also true. In a study of those with sleep disordered breathing, half of the women and 6 percent of the men were also found to have fibromyalgia. (D Germanowicz, M S Lumertz, D Martinez, and A F Margarites: Sleep disordered breathing concomitant with fibromyalgia syndrome. J Bras Pneumol, Jul. 1, 2006; 32(4): 333-8).

Although both UARS and sleep apnea are caused by blocked airflow while sleeping, there are many critical differences in the problems they cause: (Guilleminault C, Bassiri A: Clinical features and evaluation of obstructive sleep apnea-hypopnea syndrome and the upper airway resistance syndrome. In Principles and Practice of Sleep Medicine. Edn 4. Edited by Kriger M H, Roth T, Dement W C. Philadelphia: W B Saunders; 2004.)
Chronic insomnia with frequent awakenings and the inability to fall back asleep tends to be more common in patients with UARS than those with sleep apnea. (Guilleminault C, Palombini L, Poyares D, et al: Chronic insomnia, post menopausal women, and SDB, part 2: comparison of non drug treatment trials in normal breathing and UARS post menopausal women complaining of insomnia. J Psychosom Res 2002, 53:617-623.)
Patients with sleep apnea tend to fall asleep easily during the day (such as when driving), however, patients with UARS are more likely to complain of fatigue than sleepiness.
Patients with sleep apnea tend to be overweight; however, those with UARS can be any weight.
About 50 percent of with UARS are women, while only 8 percent of those with sleep apnea are female.
Upper airway resistance syndrome is often accompanied by a spastic colon and low blood pressure with lightheadedness on standing (Guilleminault C, Faul J L, Stoohs R: Sleep-disordered breathing and hypotension. Am J Respir Crit Care Med 2001, 164:1242-1247 and Guilleminault C, Khramtsov A, Stoohs R A, et al: Abnormal blood pressure in prepubertal children with sleep-disordered breathing. Pediatr Res 2004, 55:76-84.) while sleep apnea is usually associated with high blood pressure. Peppard P E, Young T, Palta M, et al: Prospective study of the association between sleep-disordered breathing and hypertension. N Engl J Med 2000, 342:1378-1384.)
People with UARS usually have cold hands and feet and other symptoms of hypothyroidism and a brainwave pattern called alpha intrusion into Delta sleep, which often occurs in CFS and fibromyalgia.

UARS is often misdiagnosed as chronic fatigue syndrome, fibromyalgia, or even ADD/hyperactivity (Gold A R, Dipalo F, Gold M S, et al: The symptoms and signs of upper airway resistance syndrome: a link to the functional somatic syndromes. Chest 2003, 123:87-95) and may be a key contributor to CFS and fibromyalgia. The sleep disorder was first recognized in children in 1982, (Guilleminault C, Winkle R, Korobkin R, et al: Children and nocturnal snoring: evaluation of the effects of sleep related respiratory resistive load and daytime functioning. Eur J Pediatr 1982, 139:165-171.), but the term UARS was not used until adult cases were reported in 1993. (Guilleminault C, Winkle R, Korobkin R, et al: Children and nocturnal snoring: evaluation of the effects of sleep related respiratory resistive load and daytime functioning. Eur J Pediatr 1982, 139:165-171).

With use of newer techniques, it has become easier to identify subtle changes in breathing patterns during sleep, and recently, UARS has been linked to not just CFS and fibromyalgia but also to attention deficit disorder and chronic insomnia.

Although a mild decrease in airflow while sleeping may not seem like a big problem, it has been shown to disrupt sleep enough to cause and/or perpetuate CFS/FMS. Therefore, keeping the airways open can be critical. (Guilleminault C, Bassiri A: Clinical features and evaluation of obstructive sleep apnea-hypopnea syndrome and the upper airway resistance syndrome. In Principles and Practice of Sleep Medicine. Edn 4. Edited by Kriger M H, Roth T, Dement W C. Philadelphia: W B Saunders; 2004. Clinical presentation of OSAS and UARS with examples of craniofacial presentations and clinical scales to define patients.)

The prior art discloses numerous devices on the market claiming to reduce airway resistance, eliminate snoring and improve the quality of sleep.

One example of a prior art device is a mechanical device sold by GlaxoSmithKline PLC under the trade mark Breathe Right™. This product is a single use, disposable device designed to reduce nasal resistance by dilating the anterior nares from the outside. It is a nasal strip which is applied to the outside of the nose to try to open the nasal passages to allow for better breathing. Each strip consists of a flexible spring-like band placed across the nose to gently lift the sides of the nose which is intended to provide nasal congestion relief due to common colds or allergies, better breathing for people with deviated septums and snoring relief.

Another device sold by Therapy Control Products Inc. under the trade mark Nozovent™, is placed inside the nostrils and stretches a portion of the lumens in the nose in an attempt to minimize nasal airflow resistance. The product claims to increase airflow up to 50% and claims to decrease snoring, help breathing and improve sleep.

In normal subjects, the nasal airway accounts for >50% of total airway resistance, with most of this contribution arising from the anterior part of the nose, including the nasal valve. The cross-sectional area of the nasal valve is controlled by several dilator muscles. In pathological conditions, nasal resistance may be considerably increased, with the extreme being total obstruction.

The upper airway has also been described as behaving like a Starling resistor. This model views the upper airway as a hollow tube with a partial obstruction at the inlet, corresponding to the nose, and a collapsible section downstream, corresponding to the oropharynx. According to this model, when air is drawn through the narrowed inlet, greater suction forces are generated downstream and may contribute to the collapsing forces that affect the collapsible segment. Thus, when nasal obstruction develops, the downstream collapsibility of the oropharynx may be increased.

In normal subjects, upper airway resistance is lower during sleep when breathing through the nose as opposed to the mouth. This contrasts with the awake state, in which the resistance has been found to be equal in a study that compared resistance between sleep and wake in the same subjects. Thus, individuals can be expected to make subconscious (automatic) efforts to breathe through the nose unless the degree of obstruction is high enough to result in an unacceptably high work of breathing. In that situation, an adaptive response may result in an automatic switch to oral-+breathing once a particular threshold of nasal airflow resistance is exceeded.

Collapse of the upper airway occurs if the negative upper airway pressure generated by inspiratory pump muscles exceeds the dilating force of these upper airway muscles. Studies that use nasal and oral flow—volume loops have indicated flow limitation via the nasal route in OSAS patients compared to normal subjects, particularly during expiration; it has also been shown that the degree of nasal flow limitation correlated with the apnoea/hypopnoea index (AHI). Positional influences may also be important, as there is evidence that supine nasal resistance is more closely related to OSA.

There is evidence of an interaction between nasal obstruction and pharyngeal narrowing in the pathophysiology of OSA. In a study of snorers, an independent relationship was found between nasal resistance and pharyngeal airspace. It has also been reported that there is a relationship between nasal obstruction, pharyngeal narrowing and AHI in subjects referred with clinical suspicion of sleep-disordered breathing (SDB). Pharyngeal narrowing was significantly related to AHI only in patients with nasal obstruction. Measurements of nasal volume using acoustic rhinometry only correlated with respiratory disturbance index measurements in non-obese subjects. These findings suggest that obesity may mask a possible independent effect of nasal obstruction in the pathophysiology of OSA.

It has also been suggested that conditions associated with variable nasal obstruction play a greater role in the pathophysiology of OSA than conditions associated with fixed obstruction. This view is at least partly based on the hypothesis that the putative adaptive response which facilitates the transition from nasal to oral breathing in conditions associated with high nasal resistance, may be more prominent in conditions associated with fixed obstruction, as the intermittent period of low nasal airflow resistance which occurs with variable nasal obstruction is likely to maintain a closer link with automatic nasal breathing. It is possible that an adaptive response associated with a switch to oral breathing limits the impact of nasal obstruction in the pathogenesis of OSA in subjects with severe fixed obstruction which may also account for the low success rate reported in surgical management of such patients.

What evidence is available supports the hypothesis that variable nasal obstruction plays a greater role in the pathophysiology of OSA than fixed obstruction. While several studies have examined the relationship between disorders associated with nasal obstruction and snoring and/or OSA, and have considered the impact of relief of nasal obstruction on OSA severity, many of these studies have taken the form of simple subjective reports, limiting the confidence in conclusions that can be drawn from them. Treatment studies of nasal obstruction that have included both subjective and objective reports of efficacy have generally reported more positive subjective responses as compared to objective responses in terms of impact on sleep-related variables. Thus, a reliable assessment of the relationship between nasal obstruction and sleep-related breathing disturbances should include objective measurements of sleep and breathing, and should ideally include objective measures of nasal obstruction and/or nasal airflow resistance.

### SUMMARY OF THE INVENTION

To this end, in one of its aspects, the invention provides a novel nasal insert adapted to be inserted into the nostrils of a person, said insert comprising a short, hollow, flexible tube with the central portion of one side being removed.

In another of its aspects, the invention provides a novel nasal insert which is adapted to convert each nasal passageway of a person from an elliptical shape to a circular shape.

In yet another of its aspects, the invention provides a novel nasal insert adapted to be inserted into the nostril of a person, said insert comprising a short, hollow, flexible tube with the central portion of one side being removed which has been impregnated with an essential oil.

### BRIEF DESCRIPTION OF THE DRAWINGS

FIG. 1 is a perspective view of the novel nasal insert of the present invention.

FIG. 2 is view of the novel nasal insert of FIG. 1 being bent prior to insertion in the nostril of a person.

FIG. 3 is view of the novel nasal insert of FIG. 1 inserted in the nostril of a person.

FIG. 4 is a schematic view of an experiment to establish the efficacy of the novel nasal insert of the present invention.

FIG. 5 is a graph showing the results of the experiment of FIG. 4.

FIG. 6 is a schematic drawing showing the area of an ellipse.

FIGS. 7A, 7B and 7C show a schematic representation of airflow through the normal shape of a nostril.

FIGS. 8A, 8B and 8C show a schematic representation of airflow through a reshaped nostril.

### DETAILED DESCRIPTION OF THE INVENTION

The upper airway of a human behaves like a Starling resistor in that obstruction at the inlet (i.e. the nasal airway) and produces collapsing forces that are manifest downstream in the collapsible segment, the pharynx.

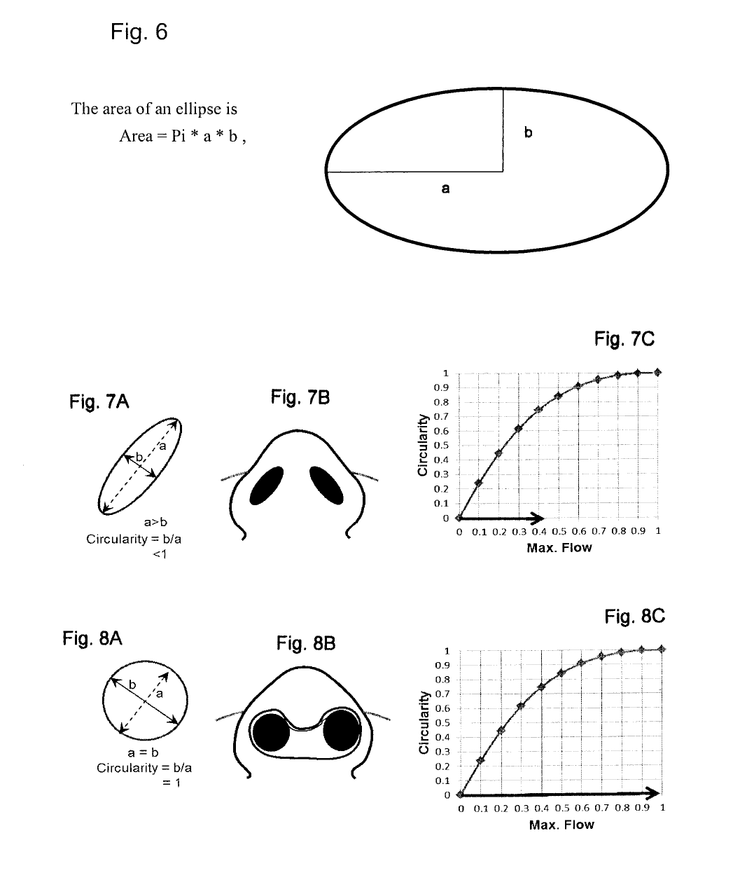

With reference to FIG. 6, the area of an ellipse is shown as per the following formula, Area=Pi*a*b. The shape of each nostril can be approximated by an ellipse. The long axis divided by the short axis is called the ellipticity and the higher the ellipticity, the further away the shape of the ellipse is from a circle, where both axes are equal. With a given pressure differential between its two ends, airflow in a tube is proportional to its cross-sectional area. The highest flow will occur in a circular pipe.

With reference to FIGS. 7 and 8, the flow through each nostril depends on its cross-sectional area. The shape of each nostril can be approximated by an ellipse. The ratio of the major axis to the minor axis (a/b) is called the ellipticity. Let the inverse of ellipticity (b/a) be called the circularity. In the case of an ellipse, the circularity varies between 0 and 1. If the circularity is 0, the ellipse is a straight line with an area of zero. If a=b, the ellipse is a circle with area=Π*a². If we change the shape of the nostril from an ellipse to a circle with the same circumference, the area will become maximal, as shown on the diagram above. The change in cross-sectional area of the nostril depends on the circularity of the original shape. It is therefore possible to predict the expected improvement in airflow by re shaping the nostril from an ellipse to a circle. The smaller the circularity of the nostril shape, the greater improvement in nasal flow is expected. Therefore, by changing the shape of the nostrils from ellipse to circle, maximum possible flow can be implemented.

To achieve maximal flow (minimal resistance) in the nose, it is therefore necessary to reshape the cross sectional area of each nasal passage along its entire length from elliptical to circular with as large a diameter as possible.

In a tapered tube, the flow is determined by the smallest cross-sectional area of the tube, so stretching only a portion of the nasal passages may increase air-flow, but not maximally.

Since many anti-snoring devices expand only a portion of the nasal passages, this may be the reason why they seem to work better for some than for others.

Unless the entire nasal passage is dilated into a circular cross sectional shape, however, maximal air flow can not be achieved. By examining the shape of the nostrils of a person, the theoretical improvement in nasal air-flow expected by re-shaping the nostrils can be easily predicted. The higher the ellipticity, the more improvement can be expected as can be seen in the graph.

The purpose of this new nasal insert is to overcome most of the shortcomings of similar devices previously available and thereby provide the lowest nasal resistance to breathing theoretically possible.

The widespread use of this device may significantly reduce the incidence of UARS, thereby elevating the productivity and well-being of millions of people that presently suffer from this serious, but treatable condition.

The nasal insert is preferably made from soft, medical grade silicone of Shore A Durameter hardness in the range 40-50. Suitable materials include “Rx-50” from Dow Corning, “Sani-Tech STHT-C” from Saint-Gobain Performance Plastics and “TYGON R-1000” also from Saint-Gobain Performance Plastics.

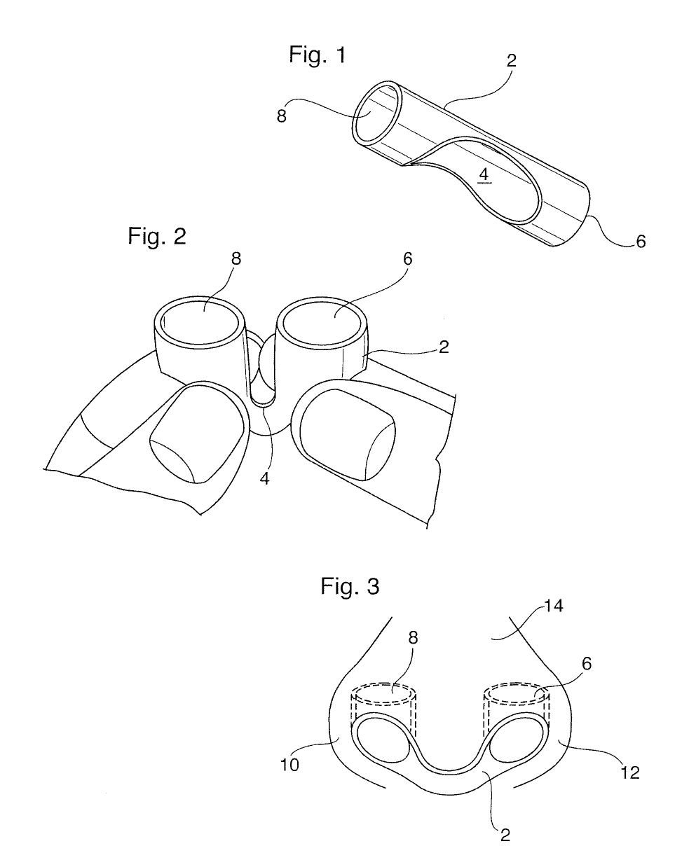

As shown in FIG. 1, the nasal insert consist of a flexible hollow tube 2 with the central area on one side of the tube removed. Ends 6,8 are at each respective end of the insert and are open.

As shown in FIG. 2, the insert is flexible and can be bent so that it may be inserted into each nostril 10, 12 of the nose 14. When inserted, the ends 6,8 are as shown in dotted lines in FIG. 3.

The structure of the novel nasal insert of the present invention creates a spring like action which when the insert is inserted into the nostrils, applies an increased pressure against the exterior inside walls of each nostril. This increased friction and pressure allows the insert to be retained in the nostrils of an individual without any exterior fixation device and prevents the insert from falling out of the nostrils of the user.

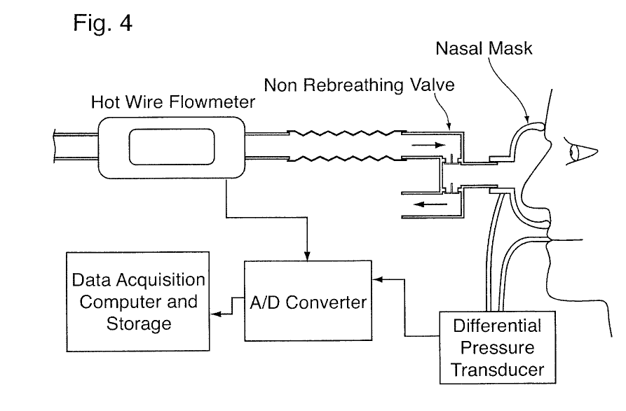

An experiment was conducted to establish the efficacy of the insert. The setup is shown on FIG. 4.

The subject breathes through a nasal mask connected to a non-rebreathing valve which separates the inspiratory flow and expiratory flow. The inspiratory side of the valve is connected to a hot-wire flowmeter (Model 4040, TSI). To protect the flow meter from humidity contamination, expiration exits through the expiratory port of the non-rebreathing valve directly to the atmosphere. The differential pressure between the inside of the mask and the interior of the subject's mouth is monitored by a differential pressure transducer (Model DP 101, Voltek Enterprises Inc.). The analog signals from the flow meter and the differential pressure transducer are digitized by an A/D converter (Model NI USB-6009, National Instruments). The digitized data is continuously gathered and recorded on the hard-drive of a laptop computer using proprietary software (Voltek Enterprises Inc.) for subsequent retrieval and analysis.

First, the subject breathes maximally through the apparatus without any nasal insert for about 10 breaths. Immediately after these control breaths, a nasal insert is placed in the nose of the subject and the same breathing pattern is repeated. The data is subsequently plotted in an Excel spreadsheet as an XY graph, with negative pressure on the Y-axis and Flow on the X-axis.

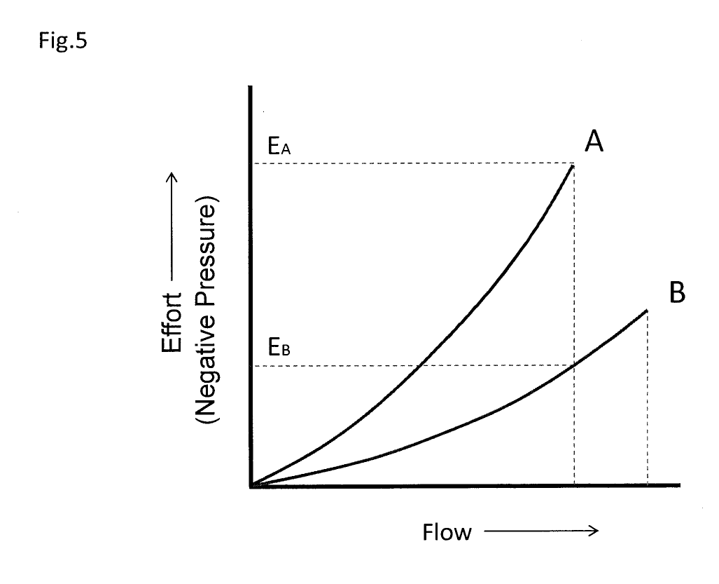

The following results were obtained and plotted on FIG. 5. Each curve represents the average of 10 breaths of a single test subject. The results show a significant difference between the inspiratory flow profiles with and without the nasal insert. The effort (negative pressure) required to provide a certain inspiratory flow is significantly reduced by the introduction of the insert, indicating that the insert reduces nasal air-flow resistance significantly. This experimental methodology may be very useful in assessing the efficacy of any nasal dilating device.

In order to improve the effectiveness of the nasal insert, it may be impregnated with an essential oil (or a combination of several essential oils) which, when inhaled from the nasal insert, will assist in opening the upper airway.

The insert may be impregnated with an essential oil by placing it a sealed chamber containing the essential oil which may be diluted with a carrier oil. The concentration is controlled by the time of exposure

It has been found that maximum efficiency is achieved when the insert has an almost undetectable odour. If the odour is too high, the desired effect may be reversed. The vapours of the following essential oils are useful for opening the sinus and breathing passages.

| NAME | BOTANICAL NAME |
|---|---|
| Allspice Leaf (Pimento Leaf) | Pimenta dioica, Pimento officinalis |
| Angelica Root | Angelica archangelica, Angelica |
| | officinalis |
| Bay (Sweet) | Laurus nobilis |
| Bay (West Indian) | Myrcia acris, Pimenta acris, Pimenta |
| | nacemosa |
| Bluegrass (African) | Cymbopogon validus |
| Camphor | Cinnamomum camphora, Laurus |
| | camphora |
| Cederwood | Juniperus virginiana |
| Clove Bud | Caryophyllus aromaticus |
| Clove Leaf | Eugenia aromatica, Eugenia |
| | caryophyllata |
| Cornmint | Mentha arvensis |
| Cubeb | Cubeba officinalis, Piper cubeba |
| Elemi | Canarium commune, Canarium luzonicum |
| Eucaliptus | Eucalyptus dives |
| Fir Balsam Needles | Abies balsamea |
| Frankincense (Olibanum) | Boswellia carter', |
| | Boswellia sacra, Boswellia thurifera |
| Gingergrass | Cymbopogon martinii var. sofia |
| Helichrisum (African) | Helichrysum splendidum |
| Khella | Ammi Visagna |
| Larch | Larix europaea |
| Lavender | Lavandula angustifolia, Lavandula |
| | officinalis |
| Magnolia (Flower) | Magnolia grandiflora, Magnolia liliflora |
| Marjoram (Spanish) | Thymus mastichina |
| Marjoram (Sweet) | Marjorana hortensis, Origanum |
| | marjorana |
| Mastic | Pistacia lentiscus |
| Mullein | Verbascum thapsus |
| Myrtle (Lemon) | Backhousia citriodona |
| Nerolina | Melaleuca quinquenervia |
| Niaoulij | Melaleuca quinquenervia, Melaleuca |
| | viridiflora |
| Oregano | Origanum vulgare |
| Peppermint | Mentha piperita |
| PeriIla | PeriIla frutescens, PeriIla ocimoides |
| Pine | Pinus sylvestris |
| Ravensara Aromatica | Cinnamonum camphora, Ravensara |
| | aromatica |
| Rosalina (Lavender Tea Tree) | Melaleuca ericifolia |
| Rosemary | Rosmarinus officinalis |
| Savory | Calamintha hortensis, Satureja montana, |
| | Satureja hortensis |
| Spearmint | Mentha spicata, Mentha viridis |
| Spruce (Black) | Picea mariana |
| Spruce (Sitka) | Picea sitchensis |
| Spruce-Hemlock | Tsuga canadensis |
| Tana | Rhus taratana |
| Tea Tree | Melaleuca alternifolia, Melaleuca |
| | linariifolia, Malaleuca uncirata |
| Terebinth | Pinus maritima, Pinus palustris, Pistacia |
| | terebinthus |
| Thyme | Thymus (aestivus, citriodorus, ilerdensis, |
| | satureiodes, valentianus, vulgaris, |
| | webbianus) |
| Yarrow | Achillea millefolium |
| Zedoary | Curcuma zedoaria |

The nasal insert of the present invention is also particularly useful for athletes. One of the problems associated with athletics is for the athlete, particularly under strenuous conditions, to be able to increase the amount of air entering through the nostrils. By using the novel nasal insert of the present invention, athletes are able to increase the amount of airflow into their nostrils thus improving their athletic performance.

Although the disclosure describes and illustrates a preferred embodiment of the invention, it is to be understood that it is not restricted to the preferred embodiment but is considered to be within the scope and spirit of the disclosure.

1. A novel nasal insert adapted to be inserted into the nostril of a person, said insert comprising a short, hollow, flexible tube with the central portion of one side being removed.

2. A novel nasal insert as claimed in claim 1 which is adapted to convert each nasal passageway from an elliptical shape to a circular shape.

3. A novel nasal insert as claimed in claim 1 which is made of a soft, medical grade silicone.

4. A novel nasal insert as claimed in claim 3 which has a Shore A Durameter hardness in the range of from 40 to 50.

5. A novel nasal insert as claimed in claim 1 which has been impregnated with an essential oil.

6. A novel nasal insert as claimed in claim 1 which has been impregnated with two or more essential oils.

7. A novel nasal insert as claimed in claim 5 wherein said essential oils are selected from the group consisting of
Allspice Leaf (Pimento Leaf)
Angelica Root
Bay (Sweet)
Bay (West Indian)
Bluegrass (African)
Camphor
Cederwood
Clove Bud
Clove Leaf
Cornmint
Cubeb
Elemi
Eucaliptus
Fir Balsam Needles
Frankincense (Olibanum)
Gingergrass
Helichrisum (African)
Khella
Larch
Lavender
Magnolia (Flower)
Marjoram (Spanish)
Marjoram (Sweet)
Mastic
Mullein
Myrtle (Lemon)
Nerolina
Niaoulij
Oregano
Peppermint
Perilla
Pine
Ravensara Aromatica
Rosalina (Lavender Tea Tree)
Rosemary
Savory
Spearmint
Spruce (Black)
Spruce (Sitka)
Spruce-Hemlock
Tana
Tea Tree
Terebinth
Thyme
Yarrow and
Zedoary.

8. A novel nasal insert as claimed in claim 6 wherein said essential oils are selected from the group consisting of
Allspice Leaf (Pimento Leaf)
Angelica Root
Bay (Sweet)
Bay (West Indian)
Bluegrass (African)
Camphor
Cederwood
Clove Bud
Clove Leaf
Commint
Cubeb
Elemi
Eucaliptus
Fir Balsam Needles
Frankincense (Olibanum)
Gingergrass
Helichrisum (African)
Khella
Larch
Lavender
Magnolia (Flower)
Marjoram (Spanish)
Marjoram (Sweet)
Mastic
Mullein
Myrtle (Lemon)
Nerolina
Niaoulij
Oregano
Peppermint
Perilla
Pine
Ravensara Aromatica
Rosalina (Lavender Tea Tree)
Rosemary
Savory
Spearmint
Spruce (Black)
Spruce (Sitka)
Spruce-Hemlock
Tana
Tea Tree
Terebinth
Thyme
Yarrow and
Zedoary.

9. A novel nasal insert for use by athletes and adapted to be inserted into the nostrils of an athlete for increasing the amount airflow into the lungs of the athlete during an athletic performance, said insert comprising a short, hollow, flexible tube with the central portion of one side being removed.
